(19) United States
(12) Patent Application Publication
(10) Pub. No.: US 2026/0167981 A1
(43) Pub. Date: Jun. 18, 2026

(54) EXCISION OF RECOMBINANT DNA FROM THE GENOME OF PLANT CELLS

(71) Applicant: BASF Agricultural Solutions US LLC, Research Triangle Park, NC (US)

(72) Inventors: Katelijn D'HALLUIN, Gent (BE); Jixiang KONG, Gent (BE)

(21) Appl. No.: 19/124,789

(22) PCT Filed: Oct. 24, 2023
(86) PCT No.: PCT/EP2023/079585

§ 371 Date: Apr. 25, 2025

(30) Foreign Application Priority Data
Oct. 26, 2022 (EP) 22203914.1

(51) Int. Cl. C12N 15/82

(52) U.S. Cl. CPC C12N 15/8231; C12N 15/8213

(57) ABSTRACT

The present invention is in the field of plant molecular biology and is directed to excision of recombinant DNA from the genome of plant somatic cells.

The present invention is in the field of plant molecular biology and is directed to excision of recombinant DNA from the genome of plant somatic cells.

### INTRODUCTION

To date, several approaches have been developed to remove transgenes following plant transformation. Such methods include: (1) Sexual crosses and segregation (Gao et al., 2016; Char et al., 2017); (2) Co-transformation using two constructs, one with the selectable marker and one with the gene of interest, to allow for subsequent removal of the selectable marker gene by genetic progeny segregation of a transgenic plant with independent insertions of each of these constructs (Komari et al., 1996); (3) Homologous recombination for excision between direct repeats (Puchta, 2000); (4) transposable element-based systems (Yoder and Goldsbrough, 1994; Gao et al., 2015); (5) several site-specific recombination systems such as Cre/lox from bacteriophage P1 (Hoess et al., 1982;), Flp/Frt from Saccharomyces cerevisiae (Cox, 1983).

Recombinases have been delivered via retransformation (Odell et al., 1990) or by crossing (Bayley et al.). These procedures are laboriously and time-consuming requiring most often screening of multiple progeny plants to recover plants where excision has taken place.

Recombinases have also been delivered by transient expression by e.g., a viral based vector (Kopertekh and Schiemann, 2005). Here also, removal takes place after the TO generation and requires screening of multiple plants.

Alternative methods have been developed based on the use of a germline-specific auto-excision vector containing a Cre recombinase gene under control of a germline-specific promoter by which marker free progeny plants were obtained (Verweire et al., 2007).

Mlynarova et al. (2006) reported the use of the microspore-specific Ntm19 promoter to drive the expression of the Cre gene. The excision of the marker gene is taking place during the microsporogenesis where the efficiency was close to 100% in tobacco seeds.

The control of the excision can be further enabled by placing the recombinase under the control of an inducible/chemical promoter, an expression system that allowed spatial and temporal control regulation by either external or intrinsic signals resulting in auto-excision of both the recombinase and the selectable marker gene placed within the excision site boundaries (Chong-Perez and Angenon, 2013).

Expression of morphogenic genes has been shown to improve plant transformation efficiency and/or to improve callus formation. However, expression in the regenerated plant often compromises the quality of the plants e.g. by leading to abnormal phenotypes or sterility.

Systems have been developed to excise morphogenic genes after transformation but before shoot regeneration to increase the production of normal, fertile plants. Morphogenic gene excision has been described using a drought-inducible Rab17 promoter driving Cre recombinase expression (Vilardell et al., 1991). Although this approach worked, the desiccation step reduced event recovery and not for all events excision was achieved (Lowe et al., 2016).

The use of developmentally regulated promoters, such as Oleosin (Ole) (Anand et al., 2017b), Globulin1 (GIb1) (Belanger and Kriz, 1991), an Early embryo response gene (End2) (Casper et al., 2005), and Lipid transfer protein 2 (Ltp2) (Kalla et al., 1994) have been used to drive Cre-mediated excision of morphogenic genes in early embryo development. Although excised events were produced, the transformation and quality event frequencies were lower presumably due to premature expression of Cre, leading to untimely excision of the morphogenic genes. The use of heat-shock inducible promoters Hsp17.7 and Hsp26, driving expression of Cre, resulted in higher frequencies of TO transformation, gene-excision and quality event recovery. These methods also allowed to excise morphogenes and the selectable marker gene at the same time (Wang, N. et al., 2020).

Besides morphogenes other examples of genes or genetic elements which introduction into the genome of a plant are beneficial in the transformation process or in early stages of regeneration but are not wanted in the regenerated plant are e.g. transposons or genes that induce double strand breaks in the genome like TALEN, CRISPR/Cas, homing endonucleases and the like.

After introduction of the intended double strand break and repair via non-homologous end joining or homologous recombination, extended expression of such genes may lead to off-target cuts and accumulation of unwanted mutations in the genome.

Therefore, there is a need in the art for precise and efficient excision of transgenes from the genome of cells, especially somatic cells, before or during regeneration of shoots and/or plants from these cells.

### DETAILED DESCRIPTION OF THE INVENTION

Surprisingly we found that the expression of one or more elements of the system for excision of genetic elements from the genome under the control of the Ntm19 promoter (Custers et al. 1997, PMB 35, 689-699) in somatic cells lead to precise and efficient excision from the genome and regeneration of shoots lacking the genetic element that was intended to be excised. It is shown, that if a Cas enzyme is comprised on the excised element that is directed to introduce genome modifications in the genome of the cell, that a high percentage of regenerated shoots is comprising the intended genome alteration.

As an example for the use of this system based on the use of the Ntm19 promoter we describe a fast procedure for removal of the nuclease components shortly after introduction of the targeted genomic modification(s). This procedure is based on auto-excision of both the Cas nuclease, the guideRNA (gRNA) and the Cre recombinase prior to T0 regeneration using a Cre recombinase under control of the Ntm19 promoter.

The approach is based on the design of a construct containing the Cas nuclease, the gRNA and the Cre recombinase flanked by lox recombination sites. A selectable marker gene is positioned outside the recombination sites.

The 1st step includes the insertion of the nuclease construct and the introduction of the targeted modification(s) in the plant genome. The 2nd step after the targeted genomic modification(s) has been accomplished, includes the removal of the inserted construct by a site-specific Cre recombinase under control of the Ntm19 promoter. Besides expression in the microspore, we showed that this promoter surprisingly also shows activity in meristematic cells, such that the site-specific recombinase can be turned on and the sequences between the excision boundaries being removed by recombination or excision in an early tissue culture phase prior to transfer of TO plants to the greenhouse without requiring extra handling, physical or chemical induction. The genome editing and the subsequent removal of the nuclease components and the recombinase are achieved shortly after each other, such that the risks for off-targeting and creation of new somatic non-inheritable mutations are highly reduced or avoided.

A first embodiment of the invention is a method for excision or deletion of one or more recombinant genetic elements from the genome of a transgenic somatic plant cell, e.g. a non-gametophytic cell the method comprising the steps of
a. Introducing into the genome of said somatic cell one or more recombinant genetic elements each comprising
   i. A first excision recognition site, and
   ii. A second excision recognition site, and between said first and second excision recognition site
   iii. A polynucleotide encoding excision components capable of excising the recombinant DNA located between said first and second excision recognition site under control of a Ntm19 promoter,
b. Expressing said excision components capable of excising the recombinant DNA located between said first and second excision recognition site, wherein said excision components recognize said excision sites and excise said recombinant DNA located between said first and second excision recognition site.

The excision or deletion of said one or more recombinant genetic element or said recombinant DNA may be achieved by e.g. recombination by site-specific recombinases, or by homologous directed repair (HDR) or non-homologous end joining (NHEJ) after introduction of double-strand breaks or nicks in the excision recognition sites of i and ii.

The excision components comprise an excision protein, and, if said excision protein is a nucleic acid guided DNA endonuclease, further comprises two guideRNAs directing said nucleic acid guided DNA endonuclease protein to said first and said second excision recognition site.

The excision recognition sites may be site specific recombination sites such as lox or att-sites, recognition sites of homing endonucleases such as LAGLIDADG type homing endonuclease, for example I-CreI or I-Sce-I, recognition sites of rare cutting restriction enzymes, PAM sites adjacent to sequences complementary to guideRNA guiding CRISPR/Cas enzymes, e.g. Cas9, Cas12a, b, c etc, CasX, CasY and the like, other enzymes having a recombinase, endonuclease or nickase activity such as, for example Zn finger proteins or TALEN fused to peptides having such activity.

Excision proteins may be site-specific recombinases. Site-specific recombinase may be selected from the group comprising FLP, Cre, SSVI, lambda Int, phi C31 Int, HK022, R, Gin, Tnl721, CinH, ParA, Tn5053, Bxbl, TP907-1 or U153.

Nucleic acid guided DNA endonucleases may be CRISPR/Cas enzymes, e.g. Cas9, Cas12a, b, c etc, CasX, CasY and the like.

Excision proteins may further be homing endonucleases, such as LAGLIDADG type homing endonuclease, for example I-CreI or I-Sce-I homing endonucleases, or rare cutting restriction enzymes, or other enzymes having a recombinase, endonuclease or nickase activity such as, for example Zn finger proteins or TALEN fused to peptides having such activity.

In a preferred embodiment, the recognition sites, if sites for the introduction of double strand breaks or nicks, are cut or nicked by the same activity, e.g. are having identical sequences, or comprising sequences sufficiently homologous to hybridize to the same guideRNA.

Preferably the recombinant genetic element is excised using a Cas system, more preferably a Cas12a system or a Cre/lox recombinase system.

The recombinant genetic elements may be introduced into the genome of the somatic plant cell by any means known in the art such as particle bombardment, protoplast electroporation, virus infection, Agrobacterium mediated transformation, magnetofection, using a repair template and a CRISPR/Cas endonuclease or nickase and the like. Preferably, it is introduced using Agrobacterium mediated transformation, e.g., A. rhizogenes or A. tumefaciens mediated transformation.

The somatic plant cell may be any somatic plant cell, preferably a dicot somatic plant cell, more preferably a soy somatic plant cell. The plant cell may be a leaf cell, a stem cell, root cell, a shoot cell, a cotyledon cell, an epicotyl cell, an embryonic cell, a callus cell, a protoplast, or a meristematic cell. Preferably the cell is a meristematic cell, more preferably the cell is a meristematic cell derived from a dicotyledonous plant, most preferably from a soy plant. The meristematic cell can be a cell of a shoot apical meristem.

Besides the polynucleotide encoding the elements necessary for excising the recombinant DNA located between the excision sites i and ii—flanked by said excision sites on both ends —, the recombinant genetic element that is introduced into the genome of the plant cell in step a. in the methods of the invention may comprise further recombinant elements that remain stably integrated in the genome of the somatic plant cell after excision. Such recombinant elements may be a gene of interest that is located outside of, and not between said first and second excision site, or is flanking said first or second excision site.

In case the first and second excision recognition sites are distinct and recognized by different nucleases or different guideRNAs guiding one or different Cas nucleases or nickases, at least one of the polynucleotides encoding such nuclease is functionally linked to a Ntm19 promoter. Preferably, in such case both polynucleotides encoding such nuclease are each functionally linked to a Ntm19 promoter.

Expressing said excision components as used herein refers to expressing said components in somatic cells. This can be expressing early tissue culture stage.

A further embodiment of the invention is a method for transient expression of a gene of interest in a somatic plant cell, e.g. a non-gametophytic cell, the method comprising the steps of
a. Introducing into the genome of said somatic cell one or more recombinant genetic elements each comprising
   i. A first excision recognition site and
   ii. A second excision recognition site, and between said first and second excision recognition site
   iii. A polynucleotide encoding excision components capable of excising the recombinant DNA located between said first and said second excision recognition site under control of a Ntm19 promoter and said gene of interest, and,
b. Expressing said excision components capable of excising the recombinant DNA located between said first and said second excision recognition site, wherein said excision components recognize said excision sites and excise said recombinant DNA located between said first and said second excision recognition site.

Transient expression as used herein may also mean temporal expression or may be transient or temporal presence of a gene of interest in the genome of the cell.

In a further embodiment, said recombinant DNA located between said first and said second excision recognition site further comprises sequences encoding at least one morphogene, and/or sequences encoding genome editing components for editing a target sequence in said somatic plant cell, or sequences encoding a selectable marker.

An additional embodiment of the invention is a method for improved regeneration of a transgenic plant, plant part, callus, plant organ from a somatic plant cell, the method comprising the steps of
a. Introducing into the genome of said somatic cell one or more recombinant genetic elements each comprising
   i. A first excision recognition site, and
   ii. A second excision recognition site, and between said first and second excision recognition site
   iii. A polynucleotide encoding excision components capable of excising the recombinant DNA located between said first and second excision recognition site under control of a Ntm19 promoter and at least one morphogene functionally linked to a promoter,
b. Expressing said excision components capable of excising the recombinant DNA located between said first and second excision recognition site.

The term “morphogene” refers to a gene or a part of a gene that when expressed in a cell is improving or enhancing the regeneration of plant organs, plant tissues or plant parts from a cell or is encoding a regulator enhancing the expression of an endogenous gene which is improving or enhancing the regeneration of plant organs, plant tissues or plant parts from a cell.

Preferably the morphogene is selected from a list comprising, more preferably from a list consisting of ESR1 (Banno et al. (2001) Plant Cell 13(12)), WIND1 (Iwase et al. (2017) Plant Cell 29(1)), WUS and ESR1 (Xu et al. (2021) Sci Adv 7(33)), WOX5 or 11 (Liu et al. (2018) Plant Cell Phys 59(4)) (Liu at al. (2014) Plant Cell 26(3)), GRF5 (Kong et al. (2020) Front Plant Sci 11), GRF-GIF (Debernardi et al. (2020) Nat Biotechnol 38(11), miRNA156 (Zhang et al. (2015) Plant Cell 27(2)), AGL15 (Zheng et al. (2013) Plant Physiol 161(4)), LEC1/LEC2 (Guo et al. (2013) PLoS One 8(8)), RKD4 (Gordon-Kamm et al. (2019) Plants 8(2)), Baby Boom (Boutilier et al. (2002) Plant Cell 14(8)) and PLT (Kareem et al (2015) Current Biology 25(8)).

In a further embodiment, the methods of the invention further comprise the step of selecting cells in which excision of the recombinant DNA between said first and second excision recognition site has taken place. In yet another embodiment, the methods according to the invention further comprise the step of regenerating shoots or plantlets from said somatic cell, and selecting shoots or plantlets in which excision of the recombinant DNA between said first and second excision recognition site has taken place.

Cells, or shoots or plantlets in which excision of the recombinant DNA between said first and second excision recognition site has taken place can be selected, for example, using molecular techniques, such as PCR techniques using primers for specific amplification of the recombinant DNA between said first and second excision recognition site as described herein in the examples; or using PCR techniques using primers flanking the first and second excision recognition site and determination based on the size of the amplified product whether said recombinant DNA has been excised; or using sequencing methods. Alternatively, cells or shoots or plantlets can be selected based on the presence or absence of a positive or of a negative selectable marker. It will be clear to the skilled person that, in order to select cells, or shoots or plantlets in which excision of the recombinant DNA between said first and second excision site has taken place, they have to be distinguished from cells in which the recombinant genetic elements were never introduced. Therefore, it is also suitable to the invention to detect genetic elements that are not between the first and second excision recognition site, such as genetic elements outside of the first and the second excision recognition site, or footprints that are left by the excision recognition sites after excision. Such genetic elements can be detected for example using molecular techniques, such as PCR techniques or using sequencing methods, or using selection or screening for expression or a gene of interest in the recombinant DNA outside of the first and second excision recognition site, e.g. selection for expression of a selectable marker, such as a marker conferring herbicide tolerance, or screening for expression of a screenable marker, such as genes causing a colour change or other visible change.

Shoots can be regenerated from somatic cells as described in the art, for example by culturing on shoot induction medium as known in the art.

Another embodiment of the invention is a method to produce a plant or shoot comprising an edit in a target sequence, said method comprising:
a. introducing into the genome of a somatic cell one or more recombinant genetic elements each comprising
   i. a first excision recognition site,
   ii. a second excision recognition site, and between said first and said second excision recognition site
   iii. a polynucleotide encoding excision components capable of excising the recombinant DNA located between said first and said second excision recognition site under control of a ntm19 promoter, and sequences encoding genome editing components for editing said target sequence
b. regenerate shoots from said somatic cell,
c. select shoots comprising said edit in said target sequence and in which excision has taken place of the recombinant DNA located between said first and said second excision recognition site, and optionally
d. grow plants from said shoots.

Further provided are seeds produced from plants produced using the methods of the invention, such as seeds comprising an edit in a target sequence.

Another embodiment provides a method for removal of genome editing components shortly after introduction of the targeted genomic modifications, said method comprising:
a. introducing into the genome of a somatic cell one or more recombinant genetic elements each comprising
   i. a first excision recognition site,
   ii. a second excision recognition site, and between said first and second excision recognition site
   iii. a polynucleotide encoding excision components capable of excising the recombinant DNA located between said first and said second excision recognition site under control of a ntm19 promoter, and sequences encoding genome editing components for editing a target sequence
b. regenerate shoots from said somatic cell,
c. select shoots comprising said edit in said target sequence and in which excision has taken place of the recombinant DNA located between said first and said second excision recognition site.

In yet another embodiment, said sequences encoding genome editing components for editing said target sequence encode a site-directed nuclease, such as nucleic acid guided DNA endonuclease, or such as a Cas nuclease and a guideRNA.

In another embodiment, said recombinant genetic element further comprises a gene of interest outside of said first and second excision recognition sites.

Said gene of interest outside of said first and second excision recognition sites can be any gene of interest, such as a selectable marker, or a screenable marker, or a gene conferring herbicide tolerance, or a gene conferring pest resistance, or a gene conferring stress tolerance, or a gene for increasing yield, or a gene improving the quality of a plant or a plant product.

In yet another embodiment, the first and the second excision recognition sites are lox sites, and wherein said excision components are the Cre-recombinase protein, whereas in another embodiment said excision components are a nucleic acid guided DNA endonuclease and one or two guideRNAs directing said nucleic acid guided DNA endonuclease protein to said first and said second excision recognition site.

One embodiment of the invention is a recombinant construct comprising
i. A first excision recognition site, and
ii. A second excision recognition site, and between said first and second excision recognition site
iii. A polynucleotide encoding excision components capable of excising the recombinant DNA located between said first and second excision recognition site under control of a Ntm19 promoter.

Said recombinant construct may further comprise between said first and second excision recognition site a polynucleotide encoding a morphogene, or a polynucleotide encoding a nucleic acid guided DNA endonuclease protein excising the recombinant DNA located between said first and second excision recognition site under control of a Ntm19 promoter.

Further embodiments of the invention are the methods and the construct as defined above, wherein said Ntm19 promoter comprises a sequence selected from the group consisting of
a) a nucleic acid molecule having the sequence of SEQ ID NO: 1, and
b) a nucleic acid molecule having a sequence with an identity of at least 80% for example at least 81%, 82%, 83%, 84%, 85%, 86%, 87%, 88%, 89%, more preferably 90%, for example at least 91%, 92%, 93%, 94%, 95%, 96%, 97%, even more preferably 98% most preferably 99% over a sequence of at least 250, 300, 400, 500, 600 preferably 700, more preferably 800, even more preferably 900 consecutive nucleic acid base pairs, most preferably the entire length of SEQ ID NO:1, and
c) a fragment of at least 100 consecutive bases, preferably at least 200, 300, 400 or 500 consecutive bases, more preferably at least 600, 700, or 800 consecutive bases, most preferably at least 900 or 950 consecutive bases of a nucleic acid molecule of a) or b) having activity as the corresponding nucleic acid molecule having the sequence of SEQ ID NO: 1, in a most preferred embodiment the fragments are fragments comprising the 3′ end of the sequence of SEQ ID NO: 1, and
d) a nucleic acid molecule which is the complement or reverse complement of any of the previously mentioned nucleic acid molecules under a) to c), and
e) a nucleic acid molecule hybridizing under conditions equivalent to hybridization in 7% sodium dodecyl sulphate (SDS), 0.5 M NaPO4, 1 mM EDTA at 50° C., preferably 55° C., more preferably 60° C., even more preferably 65° C., most preferably 68° C. with washing in 2×SSC, 0.1% SDS at 50° C. preferably 55° C., more preferably 60° C., even more preferably 65° C., most preferably 68° C. to a nucleic acid molecule comprising at least 500 600, 700, 800, 900, 950 or the complete consecutive nucleotides of SEQ ID NO:1 or the complement thereof,
   and wherein the sequences under b) to e) are directing about the same promoter activity as SEQ ID NO: 1. “About the same promoter activity” means the same tissue specificity and an expression deviating from the expression directed by SEQ ID NO: 1 by not more than 75%, preferably not more than 50%, more preferably not more than 25%, most preferably not more than 10%.

A vector comprising the recombinant construct of the invention is a further embodiment of the invention.

A cell preferably a somatic plant cell, more preferably a somatic dicot plant cell, most preferably a somatic soy cell comprising the recombinant construct, or the vector of the invention are also encompassed in this invention.

### Definitions

Abbreviations: GFP—green fluorescence protein, GUS—beta-Glucuronidase, BAP—6-benzylaminopurine; 2,4-D—2,4-dichlorophenoxyacetic acid; MS—Murashige and Skoog medium; NAA—1-naphtaleneacetic acid; MES, 2-(N-morpholino-ethanesulfonic acid, IAA indole acetic acid; Kan: Kanamycin sulfate; GA3—Gibberellic acid; Timentin™: ticarcillin disodium/clavulanate potassium, microl: Microliter.

It is to be understood that this invention is not limited to the particular methodology or protocols. It is also to be understood that the terminology used herein is for the purpose of describing particular embodiments only and is not intended to limit the scope of the present invention which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth. The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). As used herein, the word “or” means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. For clarity, certain terms used in the specification are defined and used as follows:

Antiparallel: “Antiparallel” refers herein to two nucleotide sequences paired through hydrogen bonds between complementary base residues with phosphodiester bonds running in the 5′-3′ direction in one nucleotide sequence and in the 3′-5′ direction in the other nucleotide sequence.

Antisense: The term “antisense” refers to a nucleotide sequence that is inverted relative to its normal orientation for transcription or function and so expresses an RNA transcript that is complementary to a target gene mRNA molecule expressed within the host cell (e.g., it can hybridize to the target gene mRNA molecule or single stranded genomic DNA through Watson-Crick base pairing) or that is complementary to a target DNA molecule such as, for example genomic DNA present in the host cell.

Coding region: As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′-side by the nucleotide triplet “ATG” which encodes the initiator methionine and on the 3′-side by one of the three triplets which specify stop codons (i.e., TAA, TAG, TGA). In addition to containing introns, genomic forms of a gene may also include sequences located on both the 5′- and 3′-end of the sequences which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene. The 3′-flanking region may contain sequences which direct the termination of transcription, post-transcriptional cleavage and polyadenylation.

Complementary: “Complementary” or “complementarity” refers to two nucleotide sequences which comprise antiparallel nucleotide sequences capable of pairing with one another (by the base-pairing rules) upon formation of hydrogen bonds between the complementary base residues in the antiparallel nucleotide sequences. For example, the sequence 5′-AGT-3′ is complementary to the sequence 5′-ACT-3′. Complementarity can be “partial” or “total.” “Partial” complementarity is where one or more nucleic acid bases are not matched according to the base pairing rules. “Total” or “complete” complementarity between nucleic acid molecules is where each and every nucleic acid base is matched with another base under the base pairing rules. The degree of complementarity between nucleic acid molecule strands has significant effects on the efficiency and strength of hybridization between nucleic acid molecule strands. A “complement” of a nucleic acid sequence as used herein refers to a nucleotide sequence whose nucleic acid molecules show total complementarity to the nucleic acid molecules of the nucleic acid sequence.

donor DNA molecule: As used herein the terms “donor DNA molecule”, “repair DNA molecule” or “template DNA molecule” all used interchangeably herein mean a DNA molecule having a sequence that is to be introduced into the genome of a cell. It may be flanked at the 5′ and/or 3′ end by sequences homologous or identical to sequences in the target region of the genome of said cell. It may comprise sequences not naturally occurring in the respective cell such as ORFs, non-coding RNAs or regulatory elements that shall be introduced into the target region or it may comprise sequences that are homologous to the target region except for at least one mutation, a gene edit: The sequence of the donor DNA molecule may be added to the genome or it may replace a sequence in the genome of the length of the donor DNA sequence.

Double-stranded RNA: A “double-stranded RNA” molecule or “dsRNA” molecule comprises a sense RNA fragment of a nucleotide sequence and an antisense RNA fragment of the nucleotide sequence, which both comprise nucleotide sequences complementary to one another, thereby allowing the sense and antisense RNA fragments to pair and form a double-stranded RNA molecule.

Endogenous: An “endogenous” nucleotide sequence refers to a nucleotide sequence, which is present in the genome of the untransformed plant cell.

Enhanced expression: “enhance” or “increase” the expression of a nucleic acid molecule in a plant cell are used equivalently herein and mean that the level of expression of the nucleic acid molecule in a plant, part of a plant or plant cell after applying a method of the present invention is higher than its expression in the plant, part of the plant or plant cell before applying the method, or compared to a reference plant lacking a recombinant nucleic acid molecule of the invention. For example, the reference plant is comprising the same construct which is only lacking the respective NEENA. The term “enhanced” or “increased” as used herein are synonymous and means herein higher, preferably significantly higher expression of the nucleic acid molecule to be expressed. As used herein, an “enhancement” or “increase” of the level of an agent such as a protein, mRNA or RNA means that the level is increased relative to a substantially identical plant, part of a plant or plant cell grown under substantially identical conditions, lacking a recombinant nucleic acid molecule of the invention, for example lacking the NEENA molecule, the recombinant construct or recombinant vector of the invention. As used herein, “enhancement” or “increase” of the level of an agent, such as for example a preRNA, mRNA, rRNA, tRNA, snoRNA, snRNA expressed by the target gene and/or of the protein product encoded by it, means that the level is increased 50% or more, for example 100% or more, preferably 200% or more, more preferably 5 fold or more, even more preferably 10 fold or more, most preferably 20 fold or more for example 50 fold relative to a cell or organism lacking a recombinant nucleic acid molecule of the invention. The enhancement or increase can be determined by methods with which the skilled worker is familiar. Thus, the enhancement or increase of the nucleic acid or protein quantity can be determined for example by an immunological detection of the protein. Moreover, techniques such as protein assay, fluorescence, Northern hybridization, nuclease protection assay, reverse transcription (quantitative RT-PCR), ELISA (enzyme-linked immunosorbent assay), Western blotting, radioimmunoassay (RIA) or other immunoassays and fluorescence-activated cell analysis (FACS) can be employed to measure a specific protein or RNA in a plant or plant cell. Depending on the type of the induced protein product, its activity or the effect on the phenotype of the organism or the cell may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry O H et al. (1951) J Biol Chem 193:265-275) or measuring the absorption of CBB G-250 (Bradford M M (1976) Analyt Biochem 72:248-254). As one example for quantifying the activity of a protein, the detection of luciferase activity is described in the Examples below.

Expression: “Expression” refers to the biosynthesis of a gene product, preferably to the transcription and/or translation of a nucleotide sequence, for example an endogenous gene or a heterologous gene, in a cell. For example, in the case of a structural gene, expression involves transcription of the structural gene into mRNA and—optionally—the subsequent translation of mRNA into one or more polypeptides. In other cases, expression may refer only to the transcription of the DNA harboring an RNA molecule.

Expression construct: “Expression construct” as used herein mean a DNA sequence capable of directing expression of a particular nucleotide sequence in an appropriate part of a plant or plant cell, comprising a promoter functional in said part of a plant or plant cell into which it will be introduced, operatively linked to the nucleotide sequence of interest which is—optionally—operatively linked to termination signals. If translation is required, it also typically comprises sequences required for proper translation of the nucleotide sequence. The coding region may code for a protein of interest but may also code for a functional RNA of interest, for example RNAa, siRNA, snoRNA, snRNA, microRNA, ta-siRNA or any other noncoding regulatory RNA, in the sense or antisense direction. The expression construct comprising the nucleotide sequence of interest may be chimeric, meaning that one or more of its components is heterologous with respect to one or more of its other components. The expression construct may also be one, which is naturally occurring but has been obtained in a recombinant form useful for heterologous expression. Typically, however, the expression construct is heterologous with respect to the host, i.e., the particular DNA sequence of the expression construct does not occur naturally in the host cell and must have been introduced into the host cell or an ancestor of the host cell by a transformation event. The expression of the nucleotide sequence in the expression construct may be under the control of a constitutive promoter or of an inducible promoter, which initiates transcription only when the host cell is exposed to some particular external stimulus. In the case of a plant, the promoter can also be specific to a particular tissue or organ or stage of development.

Foreign: The term “foreign” refers to any nucleic acid molecule (e.g., gene sequence) which is introduced into the genome of a cell by experimental manipulations and may include sequences found in that cell so long as the introduced sequence contains some modification (e.g., a point mutation, the presence of a selectable marker gene, etc.) and is therefore distinct relative to the naturally-occurring sequence.

Functional linkage: The term “functional linkage” or “functionally linked” is to be understood as meaning, for example, the sequential arrangement of a regulatory element (e.g. a promoter) with a nucleic acid sequence to be expressed and, if appropriate, further regulatory elements (such as e.g., a terminator or a NEENA) in such a way that each of the regulatory elements can fulfill its intended function to allow, modify, facilitate or otherwise influence expression of said nucleic acid sequence. As a synonym the wording “operable linkage” or “operably linked” may be used. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. The distance between the promoter sequence and the nucleic acid sequence to be expressed recombinantly is preferably less than 200 base pairs, especially preferably less than 100 base pairs, very especially preferably less than 50 base pairs. In a preferred embodiment, the nucleic acid sequence to be transcribed is located behind the promoter in such a way that the transcription start is identical with the desired beginning of the chimeric RNA of the invention. Functional linkage, and an expression construct, can be generated by means of customary recombination and cloning techniques as described (e.g., in Maniatis T, Fritsch E F and Sambrook J (1989) Molecular Cloning: A Laboratory Manual, 2nd Ed., Cold Spring Harbor Laboratory, Cold Spring Harbor (NY); Silhavy et al. (1984) Experiments with Gene Fusions, Cold Spring Harbor Laboratory, Cold Spring Harbor (NY); Ausubel et al. (1987) Current Protocols in Molecular Biology, Greene Publishing Assoc. and Wiley Interscience; Gelvin et al. (Eds) (1990) Plant Molecular Biology Manual; Kluwer Academic Publisher, Dordrecht, The Netherlands). However, further sequences, which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of a regulatory region for example a promoter and nucleic acid sequence to be expressed, can exist in a vector-integrated form and be inserted into a plant genome, for example by transformation.

Gene: The term “gene” refers to a region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (downstream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

Genome and genomic DNA: The terms “genome” or “genomic DNA” is referring to the heritable genetic information of a host organism. Said genomic DNA comprises the DNA of the nucleus (also referred to as chromosomal DNA) but also the DNA of the plastids (e.g., chloroplasts) and other cellular organelles (e.g., mitochondria). Preferably the terms genome or genomic DNA is referring to the chromosomal DNA of the nucleus.

Genome editing, also called gene editing, genome engineering, as used herein, refers to the targeted modification of genomic DNA in which the DNA may be inserted, deleted, modified or replaced in the genome. Genome editing may use sequence-specific enzymes (such as endonuclease, nickases, base conversion enzymes) and/or donor nucleic acids (e.g. dsDNA, oligo's) to introduce desired changes in the DNA. Sequence-specific nucleases that can be programmed to recognize specific DNA sequences include meganucleases (MGNs), zinc-finger nucleases (ZFNs), TAL-effector nucleases (TALENs) and RNA-guided or DNA-guided nucleases or nickases such as Cas9, Cpf1, CasX, CasY, C2c1, C2c3, certain Argonaut-based systems (see e.g. Osakabe and Osakabe, Plant Cell Physiol. 2015 March; 56(3):389-400; Ma et al., Mol Plant. 2016 Jul. 6; 9(7):961-74; Bortesie et al., Plant Biotech J, 2016, 14; Murovec et al., Plant Biotechnol J. 15:917-926, 2017; Nakade et al., Bioengineered Vol 8, No. 3:265-273, 2017; Burstein et al., Nature 542, 37-241; Komor et al., Nature 533, 420-424, 2016; all incorporated herein by reference). Donor nucleic acids can be used as a template for repair of the DNA break induced by a sequence specific nuclease.

An edit is a modification in a target sequence made by genome editing. Said target sequence can be any target sequence in the genome of a cell.

Heterologous: The term “heterologous” with respect to a nucleic acid molecule or DNA refers to a nucleic acid molecule which is operably linked to, or is manipulated to become operably linked to, a second nucleic acid molecule, e.g. a promoter to which it is not operably linked in nature, e.g. in the genome of a WT plant, or to which it is operably linked at a different location or position in nature, e.g. in the genome of a WT plant.

Preferably the term “heterologous” with respect to a nucleic acid molecule or DNA, e.g. an enhancer refers to a nucleic acid molecule which is operably linked to, or is manipulated to become operably linked to, a second nucleic acid molecule, e.g. a promoter to which it is not operably linked in nature.

A heterologous expression construct comprising a nucleic acid molecule and one or more regulatory nucleic acid molecule (such as a promoter or a transcription termination signal) linked thereto for example is a constructs originating by experimental manipulations in which either a) said nucleic acid molecule, or b) said regulatory nucleic acid molecule or c) both (i.e. (a) and (b)) is not located in its natural (native) genetic environment or has been modified by experimental manipulations, an example of a modification being a substitution, addition, deletion, inversion or insertion of one or more nucleotide residues. Natural genetic environment refers to the natural chromosomal locus in the organism of origin, or to the presence in a genomic library. In the case of a genomic library, the natural genetic environment of the sequence of the nucleic acid molecule is preferably retained, at least in part. The environment flanks the nucleic acid sequence at least at one side and has a sequence of at least 50 bp, preferably at least 500 bp, especially preferably at least 1,000 bp, very especially preferably at least 5,000 bp, in length. A naturally occurring expression construct—for example the naturally occurring combination of a promoter with the corresponding gene—becomes a transgenic expression construct when it is modified by non-natural, synthetic “artificial” methods such as, for example, mutagenization. Such methods have been described (U.S. Pat. No. 5,565,350; WO 00/15815). For example, a protein encoding nucleic acid molecule operably linked to a promoter, which is not the native promoter of this molecule, is considered to be heterologous with respect to the promoter. Preferably, heterologous DNA is not endogenous to or not naturally associated with the cell into which it is introduced, but has been obtained from another cell or has been synthesized. Heterologous DNA also includes an endogenous DNA sequence, which contains some modification, non-naturally occurring, multiple copies of an endogenous DNA sequence, or a DNA sequence which is not naturally associated with another DNA sequence physically linked thereto. Generally, although not necessarily, heterologous DNA encodes RNA or proteins that are not normally produced by the cell into which it is expressed.

Hybridization: The term “hybridization” as defined herein is a process wherein substantially complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tm) for the specific sequence at a defined ionic strength and pH. Medium stringency conditions are when the temperature is 20° C. below Tm, and high stringency conditions are when the temperature is 10° C. below Tm. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore, medium stringency hybridisation conditions may sometimes be needed to identify such nucleic acid molecules.

The “Tm” is the temperature under defined ionic strength and pH, at which 50% of the target sequence hybridises to a perfectly matched probe. The Tm is dependent upon the solution conditions and the base composition and length of the probe. For example, longer sequences hybridise specifically at higher temperatures. The maximum rate of hybridisation is obtained from about 16° C. up to 32° C. below Tm. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:

DNA-DNA Hybrids (Meinkoth and Wahl, Anal. Biochem., 138: 267-284, 1984):

$$Tm = 81.5 ° C . + 16.6 \times log [ Na + ] a + 0.41 \times \% [ G / Cb ] - 500 \times [ Lc ] - 1 - 0.61 \times \% formamide$$

### DNA-RNA or RNA-RNA Hybrids:

$$Tm = 79.8 + 18.5 ( log 10 [ Na + ] a ) + 0.58 ( \% G / Cb ) + 11.8 ( \% G / Cb ) 2 - 820 / Lc$$

### Oligo-DNA or Oligo-RNAd Hybrids:

For <20 nucleotides: Tm=2 (ln)
For 20-35 nucleotides: Tm=22+1.46 (ln)
a or for other monovalent cation, but only accurate in the 0.01-0.4 M range.
b only accurate for % GC in the 30% to 75% range.
c L=length of duplex in base pairs.
d Oligo, oligonucleotide; In, effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-related probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate. Another example of high stringency conditions is hybridisation at 65° C. in 0.1×SSC comprising 0.1 SDS and optionally 5×Denhardt's reagent, 100 μg/ml denatured, fragmented salmon sperm DNA, 0.5% sodium pyrophosphate, followed by the washing at 65° C. in 0.3×SSC.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

“Identity”: “Identity” when used in respect to the comparison of two or more nucleic acid or amino acid molecules means that the sequences of said molecules share a certain degree of sequence similarity, the sequences being partially identical.

Enzyme variants may be defined by their sequence identity when compared to a parent enzyme. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:
Seq A: AAGATACTG length: 9 bases
Seq B: GATCTGA length: 7 bases

Hence, the shorter sequence is sequence B.

Producing a pairwise global alignment which is showing both sequences over their complete lengths results in

| | |
|---|---|
| Seq A: | AAGATACTG- |
| | \|\|\| \|\|\| |
| Seq B: | --GAT-CTGA |

The “|” symbol in the alignment indicates identical residues (which means bases for DNA or amino acids for proteins). The number of identical residues is 6.

The “-” symbol in the alignment indicates gaps. The number of gaps introduced by alignment within the Seq B is 1. The number of gaps introduced by alignment at borders of Seq B is 2, and at borders of Seq A is 1.

The alignment length showing the aligned sequences over their complete length is 10.

Producing a pairwise alignment which is showing the shorter sequence over its complete length according to the invention consequently results in:

| | |
|---|---|
| Seq A: | GATACTG- |
| | \|\|\| \|\|\| |
| Seq B: | GAT-CTGA |

Producing a pairwise alignment which is showing sequence A over its complete length according to the invention consequently results in:

| | |
|---|---|
| Seq A: | AAGATACTG |
| | \|\|\| \|\|\| |
| Seq B: | --GAT-CTG |

Producing a pairwise alignment which is showing sequence B over its complete length according to the invention consequently results in:

| | |
|---|---|
| Seq A: | GATACTG- |
| | \|\|\| \|\|\| |
| Seq B: | GAT-CTGA |

The alignment length showing the shorter sequence over its complete length is 8 (one gap is present which is factored in the alignment length of the shorter sequence).

Accordingly, the alignment length showing Seq A over its complete length would be 9 (meaning Seq A is the sequence of the invention).

Accordingly, the alignment length showing Seq B over its complete length would be 8 (meaning Seq B is the sequence of the invention).

After aligning two sequences, in a second step, an identity value is determined from the alignment produced. For purposes of this description, percent identity is calculated by %-identity=(identical residues/length of the alignment region which is showing the respective sequence of this invention over its complete length)*100. Thus, sequence identity in relation to comparison of two amino acid sequences according to this embodiment is calculated by dividing the number of identical residues by the length of the alignment region which is showing the respective sequence of this invention over its complete length. This value is multiplied with 100 to give “%-identity”. According to the example provided above, %-identity is: for Seq A being the sequence of the invention (6/9)*100=66.7%; for Seq B being the sequence of the invention (6/8)*100=75%.

The term “Introducing”, “introduction” and the like with respect to the introduction of a donor DNA molecule in the target site of a target DNA means any introduction of the sequence of the donor DNA molecule into the target region for example by the physical integration of the donor DNA molecule or a part thereof into the target region or the introduction of the sequence of the donor DNA molecule or a part thereof into the target region wherein the donor DNA is used as template for a polymerase.

Isogenic: organisms (e.g., plants), which are genetically identical, except that they may differ by the presence or absence of a heterologous DNA sequence.

Isolated: The term “isolated” as used herein means that a material has been removed by the hand of man and exists apart from its original, native environment and is therefore not a product of nature. An isolated material or molecule (such as a DNA molecule or enzyme) may exist in a purified form or may exist in a non-native environment such as, for example, in a transgenic host cell. For example, a naturally occurring polynucleotide or polypeptide present in a living plant is not isolated, but the same polynucleotide or polypeptide, separated from some or all of the coexisting materials in the natural system, is isolated. Such polynucleotides can be part of a vector and/or such polynucleotides or polypeptides could be part of a composition and would be isolated in that such a vector or composition is not part of its original environment. Preferably, the term “isolated” when used in relation to a nucleic acid molecule, as in “an isolated nucleic acid sequence” refers to a nucleic acid sequence that is identified and separated from at least one contaminant nucleic acid molecule with which it is ordinarily associated in its natural source. Isolated nucleic acid molecule is nucleic acid molecule present in a form or setting that is different from that in which it is found in nature. In contrast, non-isolated nucleic acid molecules are nucleic acid molecules such as DNA and RNA, which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs, which encode a multitude of proteins. However, an isolated nucleic acid sequence comprising for example SEQ ID NO: 1 includes, by way of example, such nucleic acid sequences in cells which ordinarily contain SEQ ID NO:1 where the nucleic acid sequence is in a chromosomal or extrachromosomal location different from that of natural cells or is otherwise flanked by a different nucleic acid sequence than that found in nature. The isolated nucleic acid sequence may be present in single-stranded or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

Minimal Promoter: promoter elements, particularly a TATA element, that are inactive or that have greatly reduced promoter activity in the absence of upstream activation. In the presence of a suitable transcription factor, the minimal promoter functions to permit transcription.

Non-coding: The term “non-coding” refers to sequences of nucleic acid molecules that do not encode part or all of an expressed protein. Non-coding sequences include but are not limited to introns, enhancers, promoter regions, 3′ untranslated regions, and 5′ untranslated regions.

Nucleic acids and nucleotides: The terms “Nucleic Acids” and “Nucleotides” refer to naturally occurring or synthetic or artificial nucleic acid or nucleotides. The terms “nucleic acids” and “nucleotides” comprise deoxyribonucleotides or ribonucleotides or any nucleotide analogue and polymers or hybrids thereof in either single- or double-stranded, sense or antisense form. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used inter-changeably herein with “gene”, “cDNA, “mRNA”, “oligonucleotide,” and “polynucleotide”. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH2, NHR, NR2, or CN. Short hairpin RNAs (shRNAs) also can comprise non-natural elements such as non-natural bases, e.g., ionosin and xanthine, non-natural sugars, e.g., 2′-methoxy ribose, or non-natural phosphodiester linkages, e.g., methylphosphonates, phosphorothioates and peptides.

Nucleic acid sequence: The phrase “nucleic acid sequence” refers to a single or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. A “target region” of a nucleic acid is a portion of a nucleic acid that is identified to be of interest. A “coding region” of a nucleic acid is the portion of the nucleic acid, which is transcribed and translated in a sequence-specific manner to produce into a particular polypeptide or protein when placed under the control of appropriate regulatory sequences. The coding region is said to encode such a polypeptide or protein.

Oligonucleotide: The term “oligonucleotide” refers to an oligomer or polymer of ribonucleic acid (RNA) or deoxyribonucleic acid (DNA) or mimetics thereof, as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide preferably includes two or more nucleomonomers covalently coupled to each other by linkages (e.g., phosphodiesters) or substitute linkages.

Overhang: An “overhang” is a relatively short single-stranded nucleotide sequence on the 5′- or 3′-hydroxyl end of a double-stranded oligonucleotide molecule (also referred to as an “extension,” “protruding end,” or “sticky end”).

Plant: is generally understood as meaning any eukaryotic single- or multi-celled organism or a cell, tissue, organ, part or propagation material (such as seeds or fruit) of same which is capable of photosynthesis. Included for the purpose of the invention are all genera and species of higher and lower plants of the Plant Kingdom. Annual, perennial, monocotyledonous and dicotyledonous plants are preferred. The term includes the mature plants, seed, shoots and seedlings and their derived parts, propagation material (such as seeds or microspores), plant organs, tissue, protoplasts, callus and other cultures, for example cell cultures, and any other type of plant cell grouping to give functional or structural units. Mature plants refer to plants at any desired developmental stage beyond that of the seedling. Seedling refers to a young immature plant at an early developmental stage. Annual, biennial, monocotyledonous and dicotyledonous plants are preferred host organisms for the generation of transgenic plants. The expression of genes is furthermore advantageous in all ornamental plants, useful or ornamental trees, flowers, cut flowers, shrubs or lawns. Plants which may be mentioned by way of example but not by limitation are angiosperms, bryophytes such as, for example, Hepaticae (liverworts) and Musci (mosses); Pteridophytes such as ferns, horsetail and club mosses; gymnosperms such as conifers, cycads, ginkgo and Gnetatae; algae such as Chlorophyceae, Phaeophpyceae, Rhodophyceae, Myxophyceae, Xanthophyceae, Bacillariophyceae (diatoms), and Euglenophyceae. Preferred are plants which are used for food or feed purpose such as the families of the Leguminosae such as pea, alfalfa and soya; Gramineae such as rice, maize, wheat, barley, sorghum, millet, rye, triticale, or oats; the family of the Umbelliferae, especially the genus Daucus, very especially the species carota (carrot) and Apium, very especially the species Graveolens dulce (celery) and many others; the family of the Solanaceae, especially the genus Lycopersicon, very especially the species esculentum (tomato) and the genus Solanum, very especially the species tuberosum (potato) and melongena (egg plant), and many others (such as tobacco); and the genus Capsicum, very especially the species annuum (peppers) and many others; the family of the Leguminosae, especially the genus Glycine, very especially the species max (soybean), alfalfa, pea, lucerne, beans or peanut and many others; and the family of the Cruciferae (Brassicacae), especially the genus Brassica, very especially the species napus (oil seed rape), campestris (beet), oleracea cv Tastie (cabbage), oleracea cv Snowball Y (cauliflower) and oleracea cv Emperor (broccoli); and of the genus Arabidopsis, very especially the species thaliana and many others; the family of the Compositae, especially the genus Lactuca, very especially the species sativa (lettuce) and many others; the family of the Asteraceae such as sunflower, Tagetes, lettuce or Calendula and many other; the family of the Cucurbitaceae such as melon, pumpkin/squash or zucchini, and linseed. Further preferred are cotton, sugar cane, hemp, flax, chillies, and the various tree, nut and wine species.

Polypeptide: The terms “polypeptide”, “peptide”, “oligopeptide”, “polypeptide”, “gene product”, “expression product” and “protein” are used interchangeably herein to refer to a polymer or oligomer of consecutive amino acid residues.

Pre-protein: Protein, which is normally targeted to a cellular organelle, such as a chloroplast, and still comprising its transit peptide.

“Precise” with respect to the introduction of a donor DNA molecule in target region means that the sequence of the donor DNA molecule is introduced into the target region without any InDels, duplications or other mutations as compared to the unaltered DNA sequence of the target region that are not comprised in the donor DNA molecule sequence.

Primary transcript: The term “primary transcript” as used herein refers to a premature RNA transcript of a gene. A “primary transcript” for example still comprises introns and/or is not yet comprising a polyA tail or a cap structure and/or is missing other modifications necessary for its correct function as transcript such as for example trimming or editing.

Promoter: The terms “promoter”, or “promoter sequence” are equivalents and as used herein, refer to a DNA sequence which when ligated to a nucleotide sequence of interest is capable of controlling the transcription of the nucleotide sequence of interest into RNA. Such promoters can for example be found in the following public databases http://www.grassius.org/grasspromdb.html, http://mendel.cs.rhul.ac.uk/mendel.php?topic=plantprom, http://ppdb.gene.nagoya-u.ac.jp/cgi-bin/index.cgi. Promoters listed there may be addressed with the methods of the invention and are herewith included by reference. A promoter is located 5′ (i.e., upstream), proximal to the transcriptional start site of a nucleotide sequence of interest whose transcription into mRNA it controls, and provides a site for specific binding by RNA polymerase and other transcription factors for initiation of transcription. Said promoter comprises for example the at least 10 kb, for example 5 kb or 2 kb proximal to the transcription start site. It may also comprise the at least 1500 bp proximal to the transcriptional start site, preferably the at least 1000 bp, more preferably the at least 500 bp, even more preferably the at least 400 bp, the at least 300 bp, the at least 200 bp or the at least 100 bp. In a further preferred embodiment, the promoter comprises the at least 50 bp proximal to the transcription start site, for example, at least 25 bp. The promoter does not comprise exon and/or intron regions or 5′ untranslated regions. The promoter may for example be heterologous or homologous to the respective plant. A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). Suitable promoters can be derived from genes of the host cells where expression should occur or from pathogens for this host cells (e.g., plants or plant pathogens like plant viruses). A plant specific promoter is a promoter suitable for regulating expression in a plant. It may be derived from a plant but also from plant pathogens or it might be a synthetic promoter designed by man. If a promoter is an inducible promoter, then the rate of transcription increases in response to an inducing agent. Also, the promoter may be regulated in a tissue-specific or tissue preferred manner such that it is only or predominantly active in transcribing the associated coding region in a specific tissue type(s) such as leaves, roots or meristem. The term “tissue specific” as it applies to a promoter refers to a promoter that is capable of directing selective expression of a nucleotide sequence of interest to a specific type of tissue (e.g., petals) in the relative absence of expression of the same nucleotide sequence of interest in a different type of tissue (e.g., roots). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected. The term “cell type specific” as applied to a promoter refers to a promoter, which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., GUS activity staining, GFP protein or immunohistochemical staining. The term “constitutive” when made in reference to a promoter or the expression derived from a promoter means that the promoter is capable of directing transcription of an operably linked nucleic acid molecule in the absence of a stimulus (e.g., heat shock, chemicals, light, etc.) in the majority of plant tissues and cells throughout substantially the entire lifespan of a plant or part of a plant. Typically, constitutive promoters are capable of directing expression of a transgene in substantially any cell and any tissue.

Promoter specificity: The term “specificity” when referring to a promoter means the pattern of expression conferred by the respective promoter. The specificity describes the tissues and/or developmental status of a plant or part thereof, in which the promoter is conferring expression of the nucleic acid molecule under the control of the respective promoter. Specificity of a promoter may also comprise the environmental conditions, under which the promoter may be activated or down-regulated such as induction or repression by biological or environmental stresses such as cold, drought, wounding or infection.

Purified: As used herein, the term “purified” refers to molecules, either nucleic or amino acid sequences that are removed from their natural environment, isolated or separated. “Substantially purified” molecules are at least 60% free, preferably at least 75% free, and more preferably at least 90% free from other components with which they are naturally associated. A purified nucleic acid sequence may be an isolated nucleic acid sequence.

Recombinant: The term “recombinant” with respect to nucleic acid molecules, DNA or genetic elements, refers to nucleic acid molecules produced by recombinant DNA techniques. Recombinant nucleic acid molecules may also comprise molecules, which as such does not exist in nature but are modified, changed, mutated or otherwise manipulated by man. Preferably, a “recombinant nucleic acid molecule” is a non-naturally occurring nucleic acid molecule that differs in sequence from a naturally occurring nucleic acid molecule by at least one nucleic acid. A “recombinant nucleic acid molecule” may also comprise a “recombinant construct” which comprises, preferably operably linked, a sequence of nucleic acid molecules not naturally occurring in that order. Preferred methods for producing said recombinant nucleic acid molecule may comprise cloning techniques, directed or non-directed mutagenesis, synthesis or recombination techniques.

Sense: The term “sense” is understood to mean a nucleic acid molecule having a sequence which is complementary or identical to a target sequence, for example a sequence which binds to a protein transcription factor and which is involved in the expression of a given gene. According to a preferred embodiment, the nucleic acid molecule comprises a gene of interest and elements allowing the expression of the said gene of interest.

Significant increase or decrease: An increase or decrease, for example in enzymatic activity or in gene expression, that is larger than the margin of error inherent in the measurement technique, preferably an increase or decrease by about 2-fold or greater of the activity of the control enzyme or expression in the control cell, more preferably an increase or decrease by about 5-fold or greater, and most preferably an increase or decrease by about 10-fold or greater.

Small nucleic acid molecules: “small nucleic acid molecules” are understood as molecules consisting of nucleic acids or derivatives thereof such as RNA or DNA. They may be double-stranded or single-stranded and are between about 15 and about 30 bp, for example between 15 and 30 bp, more preferred between about 19 and about 26 bp, for example between 19 and 26 bp, even more preferred between about 20 and about 25 bp for example between 20 and 25 bp. In an especially preferred embodiment, the oligonucleotides are between about 21 and about 24 bp, for example between 21 and 24 bp. In a most preferred embodiment, the small nucleic acid molecules are about 21 bp and about 24 bp, for example 21 bp and 24 bp.

Substantially complementary: In its broadest sense, the term “substantially complementary”, when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, more desirably at least 70%, more desirably at least 80% or 85%, preferably at least 90%, more preferably at least 93%, still more preferably at least 95% or 96%, yet still more preferably at least 97% or 98%, yet still more preferably at least 99% or most preferably 100% (the latter being equivalent to the term “identical” in this context). Preferably identity is assessed over a length of at least 19 nucleotides, preferably at least 50 nucleotides, more preferably the entire length of the nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J Mol. Biol. 48: 443-453; as defined above). A nucleotide sequence “substantially complementary” to a reference nucleotide sequence hybridizes to the reference nucleotide sequence under low stringency conditions, preferably medium stringency conditions, most preferably high stringency conditions (as defined above).

“Target site” as used herein means the position in the genome at which a double strand break or one or a pair of single strand breaks (nicks) are induced using recombinant technologies such as Zn-finger, TALEN, restriction enzymes, homing endonucleases, RNA-guided nucleases, RNA-guided nickases such as CRISPR/Cas nucleases or nickases and the like.

Transgene: The term “transgene” as used herein refers to any nucleic acid sequence, which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence.

Transgenic: The term transgenic when referring to an organism means transformed, preferably stably transformed, with a recombinant DNA molecule that preferably comprises a suitable promoter operatively linked to a DNA sequence of interest.

Vector: As used herein, the term “vector” refers to a nucleic acid molecule capable of transporting another nucleic acid molecule to which it has been linked. One type of vector is a genomic integrated vector, or “integrated vector”, which can become integrated into the chromosomal DNA of the host cell. Another type of vector is an episomal vector, i.e., a nucleic acid molecule capable of extra-chromosomal replication. Vectors capable of directing the expression of genes to which they are operatively linked are referred to herein as “expression vectors”. In the present specification, “plasmid” and “vector” are used interchangeably unless otherwise clear from the context. Expression vectors designed to produce RNAs as described herein in vitro or in vivo may contain sequences recognized by any RNA polymerase, including mitochondrial RNA polymerase, RNA pol I, RNA pol II, and RNA pol III. These vectors can be used to transcribe the desired RNA molecule in the cell according to this invention. A plant transformation vector is to be understood as a vector suitable in the process of plant transformation.

Wild-type: The term “wild-type”, “natural” or “natural origin” means with respect to an organism, polypeptide, or nucleic acid sequence, that said organism is naturally occurring or available in at least one naturally occurring organism which is not changed, mutated, or otherwise manipulated by man.

### FIGURES

FIG. 1 and FIG. 1B

For the Agro strain GA00804 [(EHA105(pTiEHA105) (pBas05397)], the InDel % distribution in T0 plants is shown in FIG. 1 and the InDel % distribution grouped by 2m-EPSPS Copy Number in FIG. 1B.

FIG. 2 and FIG. 2B

For the Agro strain GA00817 [(SHA017(pRi1599)(pBas05397)], the InDel % distribution in TO plants is shown in FIG. 2 and the InDel % distribution grouped by 2m-EPSPS Copy Number in FIG. 2B.

FIG. 3

Construct design for the excision of the Cas nuclease components. The excision construct contains the DNA fragment to be excised, consisting of the Cas12a nuclease gene, the sgRNA and the Cre-recombinase under control of the pNtm19 promoter. This DNA fragment to be excised is flanked by two directly oriented loxP recombination sites. After excision only the 2mepsps gene remains inserted in the genome. Dotted line: genomic sequence flanking the transgene insert.

### EXAMPLES

### Chemicals and Common Methods

Unless indicated otherwise, cloning procedures carried out for the purposes of the present invention including restriction digest, agarose gel electrophoresis, purification of nucleic acids, Ligation of nucleic acids, transformation, selection and cultivation of bacterial cells were performed as described (Sambrook et al., 1989). Sequence analyses of recombinant DNA were performed with a laser fluorescence DNA sequencer (Applied Biosystems, Foster City, CA, USA) using the Sanger technology (Sanger et al., 1977). Unless described otherwise, chemicals and reagents were obtained from Sigma Aldrich (Sigma Aldrich, St. Louis, USA), from Promega (Madison, WI, USA), Duchefa (Haarlem, The Netherlands) or Invitrogen (Carlsbad, CA, USA). Restriction endonucleases were from New England Biolabs (Ipswich, MA, USA). Oligonucleotides were synthesized by Integrated DNA Technologies (Coralville, IA, USA).

### Example 1: Soy Transformation

Mature seeds of Thorne were used for stable transformation. Seeds were surface sterilized in a desiccator for about 16 hrs using chlorine gas as described by Di et al. 1996. Agrobacterium transformation using half seed explants was essentially as described by Paz et al. (2006) and Luth et al. (2015). The disarmed A. tumefaciens strain EHA105 (Hood et al. 1993) and the disarmed A. rhizogenes strain SHA017 [K599(pRi2659)](Mankin et al., 2007; WO 2006/024509), both harboring the T-DNA vector pBas05397 were used for co-cultivation of the half seed explants. After 5 to 6 says co-cultivation, we selected for glyphosate resistant shoots on a selection medium containing 0.075 mM Glyphosate.

### Example 2: Plant Transformation Vector

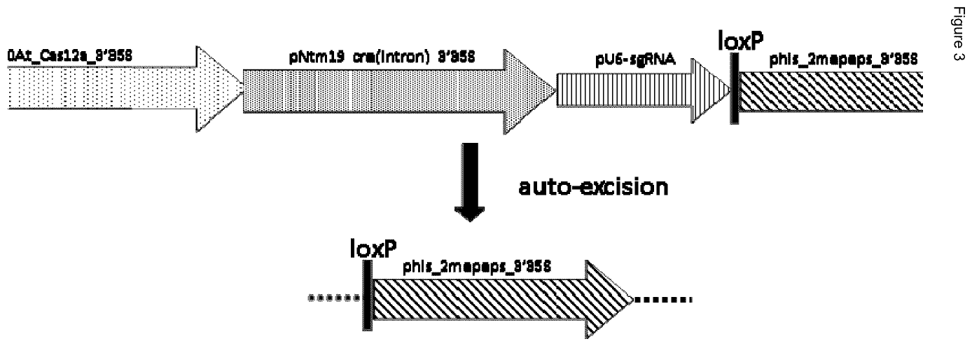

The plant transformation vector pBas05397 contains between the lox sites a Cas12a nuclease expression cassette, a gRNA cassette and a Cre-recombinase cassette, and outside the lox sites a 2m-EPSPS gene expression cassette (FIG. 3). The Cas12a expression cassette comprises a soybean codon-optimized type V CRISPR-associated protein Cas12a gene from Lachnospiraceae bacterium ND2006, driven by the promoter from the ubiquitin 10 (UBQ10) gene of Arabidopsis thaliana (Grefen et al., 2010) and a 3′35S terminator. The Cre recombinase is under control of the Nicotiana tabacum Ntm19 promoter (Oldenhof et al., 1996) and the 3′35S terminator. The gRNA cassette comprises a gRNA under control of the promoter of the small nuclear RNA (U6-10) gene of Glycine max (Sun et al. 2015) and is designed for guiding the Lb Cas12a nuclease to the FAD2 Target Sequence (TS) TTTA-GTCCCTTATTTCTCATGGAAAAT (SEQ ID NO: 2). In the gRNA cassette, the sequence encoding the spacer RNA was between direct repeats (DRs) (Zetsche et al., 2016) and preceded by a truncated tRNA at the 5′end (RNA of the glycine transfer RNA gene of Triticum aestivum (Marcu K B. et al., 1977)). The T-DNA vector pBas05397 contains further a 2m-EPSPS gene expression cassette with the 2m-EPSPS gene under control of the Arabidopsis thaliana Ph4a748_ABC histon promoter and the 3′his terminator to allow for selection on glyphosate. The plant transformation vector pBas05019 is identical to pBas05397 but contains no Cre recombinase and no lox sites.

### Example 3: Genotyping of Transformants

For genotyping individual soy transformants, a piece of a top leaf of an “in vitro” shoot of about 10 cm in size was harvested in 1 mL tubes on a 96-well plate for genomic DNA extraction.

Copy number of the 2m-EPSPS gene was determined by real-time PCR (Ingham et al., 2001). InDel efficiencies were measured using a ddPCR drop-off assay. ddPCR assays were designed using Primer3Plus software with modified settings compatible with the applied master mix. To avoid loss of binding sites, primers and reference probe were designed away from the cut site. PCR primers were designed according to the following guidelines: primer length of 17-24 bases, primer melting temperature of 55 to 60° C. with an ideal temperature of 58° C., melting temperatures of the two primers differ by no more than 2° C., primer GC content of 35-65%, amplicon size of 100-250 bases. Drop-off probes were designed to lose their binding site when one or more base substitutions, insertions or deletions were introduced at the FAD2 Target site. The sequences of the probes and primers are shown in Table 1.

TABLE 1
Primers and probes used for the ddPCR drop-off assay to determine
editing frequencies at the FAD2 target site.

| Primer/probe type | Sequence | SEQ ID NO |
|---|---|---|
| Forward Primer | TGATTGCTCACGAGTGT | 3 |
| Reverse Primer | CAGAGACATTGAAGGCTAAA | 4 |
| Reference probe | CCGTGATGAAGTGTTTGTCCCA | 5 |
| (FAM) | | |
| Drop-off probe (HEX) | CTCATGGAAAATAAGCCATCG | 6 |

To screen for “in vitro” shoots with the Cas12a nuclease being removed by the Cre/lox recombinase under control of the Ntm19 promoter, shoots were further analyzed by PCR using primer set (HT-19-001 Forward/HT-22-007 Reverse) for specific amplification of the Cas12a nuclease (Table 2).

TABLE 2
Primers used for PCR to screen shoots for
presence or absence of Cas12a.

| | | SEQ ID |
|---|---|---|
| Primer | Sequence | NO |
| HT-19-001 | AAGAAGAAGAGGAAGGTGGG | 7 |
| HT-22-007 | GAGAGGTCAGCATCAGCATAC | 8 |

To identify the different types of mutations and their frequency at which the different types of mutations had occurred at the FAD2 target site, NGS was performed. The region surrounding the target site was PCR amplified with Q5 High-Fidelity polymerase (M0492L) using the primer pair HT-21-080 Forward/HT-21-081 Reverse to amplify a region of 354 bp (Table 3).

TABLE 3
Primer pair to amplify a target region of
354 bp for NGS.

| | | SEQ ID |
|---|---|---|
| Primer | Sequence | NO |
| HT-21-080 | CCTCATTGCATGGCCAATC | 9 |
| HT-21-081 | CCAGAGACATTGAAGGCTAAATAC | 10 |

### Example 4: Soy Genome Editing by Removal of the Nuclease Components in the Tissue Culture Phase

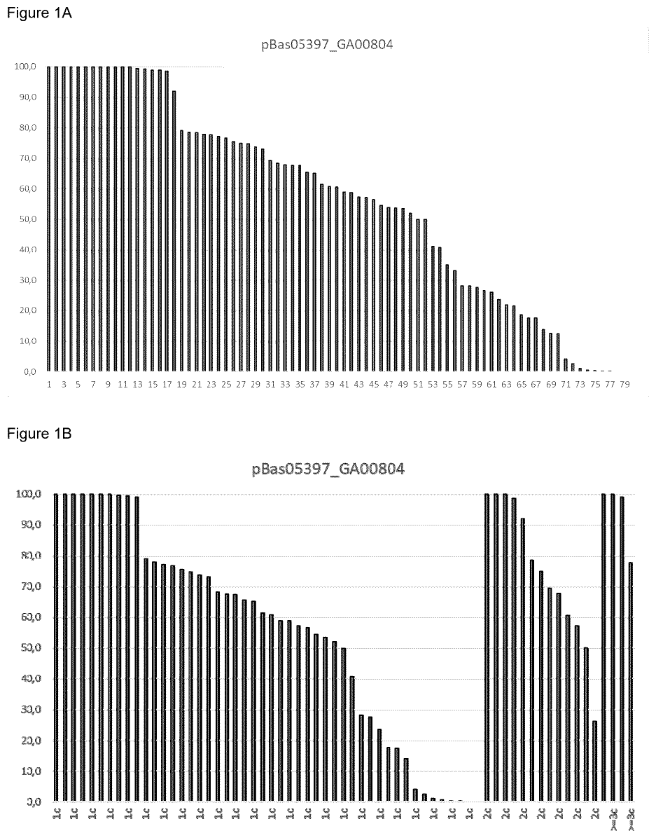

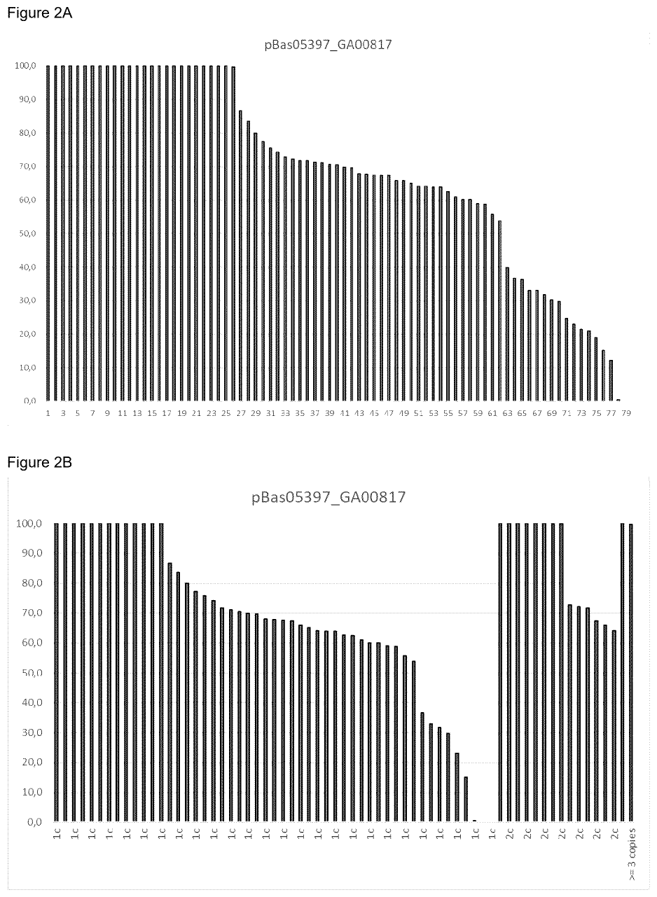

Half seeds were co-cultivated with the disarmed A. tumefaciens strain GA00804 (EHA105 (pTiEHA105)(pBas05397)) or the disarmed A. rhizogenes strain GA00817 (SHA017(pRi2659)(pBas05397)). For each strain, 80 independent glyphosate resistant shoots were analyzed for determination of InDel % at the FAD2 Target Site by ddPCR drop-off % and for determination of copy number of the 2m-EPSPS gene by real time PCR (FIGS. 1A, 1B and 2A, 2B). Single copy 2m-EPSPS events were obtained at frequencies of 60% and 67% with the Agro strains GA00804 and GA00817, respectively (FIGS. 1B and 2B).

To screen for “in vitro” shoots with the Cas12a nuclease being removed by the Cre/lox recombinase under control of the Ntm19 promoter, shoots were further analyzed by PCR using primer set (HT-19-001 Forward/HT-22-007 Reverse) for specific amplification of the Cas12a nuclease. PCR analysis for presence or absence of the Cas12a nuclease using primer set (HT-19-001 Forward/HT-22-007 Reverse) on transformants with the GA00804 or GA00817 strains, showed that 33% (26 out of 80 transformants with GA00804) and 40% (32 out of 80 with GA00817) didn't yield a PCR product, indicating that the nuclease components have been removed. With the transformation vector pBas05019 that is identical to transformation vector pBAS05397 but without the Cre recombinase and lox sites, 99% (158/160) of the transformants contained still the Cas12a nuclease.

NGS analysis has been performed on a subset of 1 copy 2m-EPSPS GA00804 and GA00817 transformants from which the Cas12a nuclease components have been removed by Cre/lox recombination. We assessed for each specific mutation at the FAD2 target locus the mutation frequency by calculating the percentage of sequence reads with a specific mutation (deletion) as a proportion of the total number of reads. These data are summarized in Table 4. Mutations were mostly deletions. For example, event TMGM0139-058-01$001 has 4 types of deletions of 8, 12, 14 and 22 nucleotides occurring at frequencies of 24.7, 21.5, 26.4 and 26.8%, respectively. The control event TMGM0139-Ctrl006-01$001 derived from a half seed explant not co-cultivated with Agrobacterium showed WT reads at a frequency of ˜100%, as expected. Table 4 further shows that the editing frequencies obtained by ddPCR drop-off correlate very well with those obtained by NGS demonstrating that our assays are reliable.

These data show that the nuclease components can be removed in an early tissue culture phase prior to transfer of TO plants to the greenhouse by Cre/lox recombination with the Cre recombinase under control of the Ntm19 promoter, such that the creation of new somatic non-inheritable mutations during later developmental stages can be avoided.

TABLE 4
Percent (%) reads for each specific mutation at the FAD2 Target Sites for 1copy
2m-EPSPS events from which the Cas12a nuclease components have been removed by
Cre/lox recombination by a Cre recombinase under control of the Ntm19 promoter.

| | | ddPCR | NGS | | | |
|---|---|---|---|---|---|---|
| | | % drop | no | % | % | type of |
| | Ceresname | off | reads | WT | INDELS | deletion (%) |
| GA00804: | TMGM0136-008-01$001 | 61.40 | 280502 | 38.68 | 59.2 | 9 (17.9); 7 (41.4) |
| pNtm19 | TMGM0136-041-01$001 | 68.26 | 271346 | 24.93 | 74.91 | 47 (24.2); 46 (26.5); |
| cre | | | | | | 11 (16.71); +14 (7.5) |
| | TMGM0136-044-01$001 | 99.36 | 168186 | 0.07 | 99.91 | 41 (32.85); 24 (29.47); |
| | | | | | | 11 (37.57) |
| | TMGM0136-046-01$001 | 74.73 | 287854 | 17.77 | 80.65 | 55 (31.3); 14 (23.9); |
| | | | | | | 12 (13.12); 5 (12.36) |
| | TMGM0136-058-01$001 | 65.41 | 216964 | 31.31 | 68.54 | 32 (21.4); 17 (15.9); |
| | | | | | | 7 (31.2) |
| | TMGM0136-060-01$001 | 51.95 | 253420 | 46.97 | 52.54 | 14 (15.5); 13 (15.0); |
| | | | | | | 10 (10.9); 7 (11.2) |
| | TMGM0136-068-01$001 | 67.50 | 173934 | 30.83 | 69.18 | 20 (26.6); 14 (16.5); |
| | | | | | | 7 (25.3) |
| | TMGM0136-075-01$001 | 73.66 | 302218 | 24.78 | 74.32 | 9 (58.7); 7 (15.7) |
| | TMGM0136-079-01$001 | 75.43 | 298970 | 20.66 | 77.98 | 16 (26.9); 12 (19.8); |
| | | | | | | 9 (13); 7 (15.5) |
| GA00817: | TMGM0139-002-01$001 | 53.78 | 234304 | 40.01 | 60.03 | 10 (15); 11 (15.8); |
| pNtm19 | | | | | | 13 (14.2); 18 (14.9) |
| cre | TMGM0139-005-01$001 | 60.03 | 142928 | 41.5 | 58.5 | 7 (15.3); 10 (14.3); |
| | | | | | | 12 (15.3); 13 (13.7) |
| | TMGM0139-011-01$001 | 29.60 | 308746 | 64.47 | 35.5 | 6 (8.9); 12 (9.2); |
| | | | | | | 14 (17.3) |
| | TMGM0139-015-01$001 | 99.96 | 282762 | 0 | 100 | 6 (7.4); 10 (8.7); |
| | | | | | | 11 (17.5); 12 (15.1); |
| | | | | | | 13 (8.8); 14 (34); |
| | | | | | | 17 (8.2) |
| | TMGM0139-023-01$001 | 67.30 | 267516 | 31.82 | 68.12 | 5 (17.5); 7 (15.5); |
| | | | | | | 10 (17.8); 14 (17.1) |
| | TMGM0139-036-01$001 | 100.00 | 316090 | 0 | 100 | 8 (22.6); 10 (23.2); |
| | | | | | | 16 (23.6); 17 (27.1) |
| | TMGM0139-053-01$001 | 99.99 | 396372 | 0 | 100 | 5 (21.8); 7 (22.9); |
| | | | | | | 14 (54.3) |
| | TMGM0139-057-01$001 | 99.99 | 312766 | 0 | 100 | 10 (26.1); 11 (26.3); |
| | | | | | | 12 (22.8); 16 (24.4) |
| | TMGM0139-058-01$001 | 99.98 | 346712 | 0 | 100 | 8 (24.7); 12 (21.5); |
| | | | | | | 14 (26.4); 22 (26.8) |
| | TMGM0139-059-01$001 | 100.00 | 257658 | 0 | 100 | 7 (21.6); 10 (50.8); |
| | | | | | | 21 (27.4) |
| | TMGM0139-076-01$001 | 67.94 | 288470 | 28.01 | 71.61 | 7 (12.4); 13 (15.4); |
| | | | | | | 39 (20.9); 93 (22.4) |
| | TMGM0139-Ctrl006-01$001 | 0.00 | 270792 | 99.98 | 0.01 | 11 (0.01) = |
| | | | | | | Background |

Edited plants lacking the Cas12a nuclease were transferred to the greenhouse for seed production by selfing and evaluation of the inheritance of the edits in the T1 progeny.

### Example 5: Oilseed Rape Genome Editing by Removal of the Nuclease Components in the Tissue Culture Phase

In another experiment, the removal of the nuclease components in the tissue culture phase is tested in oilseed rape (Brassica napus) protoplasts. Oilseed rape protoplasts are isolated from the leaves of 4- to 7-week-old aseptically grown plants. Healthy leaves are cut into fine strips with a sharp razor blade. The strips are infiltrated with cell wall-dissolving enzyme solution (1.5% cellulase R10 and 0.75% macerozyme R10 in 10 mM KCl and 0.6 M mannitol, pH 7.5) and incubated overnight in the dark with gentle shaking (40 rpm) at 24° C. After enzymatic digestion, the released protoplasts are collected by filtering the mixture through 40-μm nylon meshes and resuspended in W5 solution. The resuspended protoplasts are kept on ice and allowed to settle by gravity, after which the cell pellet is resuspended in MMG. For transformation, 200 μl of cells (2.5×105) are mixed with 20 μg plasmid DNA and 220 μl of freshly prepared polyethylene glycol (PEG) solution. The mixture is incubated for 15-20 min in the dark. After removing the PEG solution, the protoplasts are embedded in alginate layers as described by Kielkowska and Adamus (2012) and incubated at ˜25° C. Protoplast-derived colonies are picked and transferred to shoot induction medium (Murashige & Skoog, 1962) with a high cytokinin/auxin ratio i.e., 1 mg/L BAP+0.1 mg/L NAA or BAP or 3 mg/L BAP+0.1 mg/L NAA+0.1 mg/L GA3 with selection on glyphosate. Further plant regeneration is performed as described by De Block et al. (1989).

The plasmid DNA that is used for transformation contains between the lox sites a Cas12a nuclease expression cassette, a gRNA cassette and a Cre-recombinase cassette, and outside the lox sites a 2m-EPSPS gene expression cassette. The Cas12a expression cassette comprises a type V CRISPR-associated protein Cas12a gene, driven by the promoter from the ubiquitin 10 (UBQ10) gene of Arabidopsis thaliana (Grefen et al., 2010) and a 3′35S terminator. The Cre recombinase is under control of the Nicotiana tabacum Ntm19 promoter (Oldenhof et al., 1996) and the 3′35S terminator. The gRNA cassette comprises a gRNA under control of the polymerase III-type promoter of the Arabidopsis U6 snRNA gene and is designed for guiding the Cas12a nuclease to the BnFAD2 Target Sequence. In the gRNA cassette, the sequence encoding the spacer RNA is between direct repeats (DRs) (Zetsche et al., 2016) and preceded by a truncated tRNA at the 5′end (RNA of the glycine transfer RNA gene of Triticum aestivum (Marcu K B. et al., 1977)). The plasmid contains further a 2m-EPSPS gene expression cassette with the 2m-EPSPS gene under control of the Arabidopsis thaliana Ph4a748_ABC histon promoter and the 3′his terminator to allow for selection on glyphosate. As a control, a plasmid is used which is identical to the plasmid but contains no Cre recombinase and no lox sites.

For the transfected plasmid and control plasmid, independent glyphosate resistant shoots are analyzed for determination of InDel % at the FAD2 Target Site and for determination of copy number of the 2m-EPSPS gene.

To screen for “in vitro” shoots with the Cas12a nuclease being removed by the Cre/lox recombinase under control of the Ntm19 promoter, shoots are further analyzed by PCR using a primer set for specific amplification of the Cas12a nuclease. PCR analysis for presence or absence of the Cas12a nuclease using a primer set shows that transformants can be obtained that do not yield a PCR product, indicating that the nuclease components are removed. With the control plasmid, all the transformants contain still the Cas12a nuclease.

1. A method for excision of one or more recombinant genetic elements from the genome of a transgenic somatic plant cell, the method comprising the steps of
a. introducing into the genome of said somatic cell one or more recombinant genetic elements each comprising
i. a first excision recognition site,
ii. a second excision recognition site, and between said first and second excision recognition site
iii. a polynucleotide encoding excision components capable of excising the recombinant DNA located between said first and second excision recognition site under control of a ntm19 promoter, and
b. expressing said excision components capable of excising the recombinant DNA located between said first and second excision recognition site, wherein said excision components recognize said excision sites and excise said recombinant DNA located between said first and second excision recognition site.

2. A method for transient expression of a gene of interest in a somatic plant cell, the method comprising the steps of
a. introducing into the genome of said somatic cell one or more recombinant genetic elements each comprising
i. a first excision recognition site,
ii. a second excision recognition site, and between said first and second excision recognition site
iii. a polynucleotide encoding excision components capable of excising the recombinant DNA located between said first and said second excision recognition site under control of a ntm19 promoter, and said gene of interest, and,
b. expressing said excision components capable of excising the recombinant DNA located between said first and said second excision recognition site, wherein said excision components recognize said excision sites and excise said recombinant DNA located between said first and said second excision recognition site.

3. The method of claim 1, wherein said recombinant DNA located between said first and said second excision recognition site further comprises sequences encoding at least one morphogene, and/or sequences encoding genome editing components for editing a target sequence in said somatic plant cell, and/or sequences encoding a selectable marker.

4. A method for improved regeneration of a transgenic plant from a somatic plant cell, the method comprising the steps of
a. introducing into the genome of said somatic cell one or more recombinant genetic elements each comprising
i. a first excision recognition site, and
ii. a second excision recognition site, and between said first and second excision recognition site
iii. a polynucleotide encoding excision components capable of excising the recombinant DNA located between said first and second excision recognition site under control of a ntm19 promoter and at least one morphogene functionally linked to a promoter, and
b. expressing said excision components capable of excising the recombinant DNA located between said first and second excision recognition site, wherein said excision components recognize and excise said recombinant DNA located between said first and second excision recognition site.

5. The method of claim 1, further comprising the step of selecting cells in which excision of the recombinant DNA between said first and second excision recognition site has taken place.

6. The method of claim 1, further comprising the step of regenerating shoots or plantlets from said somatic cell, and selecting shoots or plantlets in which excision of the recombinant DNA between said first and second excision recognition site has taken place.

7. A method to produce a plant or shoot comprising an edit in a target sequence, said method comprising:
a. introducing into the genome of a somatic cell one or more recombinant genetic elements each comprising
i. a first excision recognition site,
ii. a second excision recognition site, and between said first and said second excision recognition site
iii. a polynucleotide encoding excision components capable of excising the recombinant DNA located between said first and said second excision recognition site under control of a ntm19 promoter, and sequences encoding genome editing components for editing said target sequence
b. regenerate shoots from said somatic cell,
c. select shoots comprising said edit in said target sequence and in which excision has taken place of the recombinant DNA located between said first and said second excision recognition site, and optionally
d. grow plants from said shoots.

8. A method for removal of genome editing components shortly after introduction of the targeted genomic modifications, said method comprising:
a. introducing into the genome of a somatic cell one or more recombinant genetic elements each comprising
i. a first excision recognition site,
ii. a second excision recognition site, and between said first and second excision recognition site
iii. a polynucleotide encoding excision components capable of excising the recombinant DNA located between said first and said second excision recognition site under control of a ntm19 promoter, and sequences encoding genome editing components for editing a target sequence
b. regenerate shoots from said somatic cell, and
c. select shoots comprising said edit in said target sequence and in which excision has taken place of the recombinant DNA located between said first and said second excision recognition site.

9. The method of claim 7, wherein said sequences encoding genome editing components for editing said target sequence encode a site-directed nuclease.

10. The method of claim 1, wherein said recombinant genetic element further comprises a gene of interest outside of said first and second excision recognition sites.

11. The method according to claim 1, wherein the first and the second excision recognition sites are lox sites, and wherein said excision components are the Crerecombinase protein.

12. The method according to claim 1, wherein said excision components are a nucleic acid guided DNA endonuclease and one or two guideRNAs directing said nucleic acid guided DNA endonuclease protein to said first and said second excision recognition site.

13. A recombinant construct comprising
i. a first excision recognition site, and
ii. a second excision recognition site, and between said first and second excision recognition site
iii. a polynucleotide encoding excision components capable of excising the recombinant DNA located between said first and second excision recognition site under control of a ntm19 promoter.

14. The method of claim 1, wherein said ntm19 promoter comprises a sequence selected from the group consisting of
a) a nucleic acid molecule having the sequence of SEQ ID NO: 1, and
b) a nucleic acid molecule having a sequence with an identity of at least 80% to SEQ ID NO:1, and
c) a fragment of at least 100 consecutive bases of a nucleic acid molecule of I) or II) having activity as the corresponding nucleic acid molecule having the sequence of SEQ ID NO: 1, and
d) a nucleic acid molecule which is the complement or reverse complement of any of the previously mentioned nucleic acid molecules under I) to III), and
e) a nucleic acid molecule hybridizing under conditions equivalent to hybridization in 7% sodium dodecyl sulphate (SDS), 0.5 M NaPO4, 1 mM EDTA at 50° C. with washing in 2×SSC, 0.1% SDS at 50° C. to a nucleic acid molecule comprising at least 50 consecutive nucleotides of SEQ ID NO:1 or the complement thereof.

15. A vector comprising the recombinant construct of claim 13.

16. A cell comprising the recombinant construct of claim 13.

17. The method of claim 9, wherein the site-directed nuclease is a nucleic acid guided DNA endonuclease.

18. The method of claim 9, wherein the sequences encoding genome editing components for editing said target sequence encode a Cas nuclease and a guideRNA.

19. A vector comprising the recombinant construct of claim 14.

20. A cell comprising the recombinant construct of claim 14.


SEQUENCE LISTING (10 sequences)

SEQ ID NO: 1 - DNA, 986 bp, Nicotiana tabacum
  source 1..986: mol_type = genomic DNA
  misc_feature 1..986: note = Pntm19-1.2
agatgtatagggtcctaatgcgggtactgaacaccaggtgggaaacaaaaaatatacagaacaactcctttagaatttacaatttttgagcgtgttggcttggtacgattctacttttcatatctctcgtcatctcctaactcctatggttcaccagccaccgattaattatgacaccgctaacaaaaatcttgcgacgacattgagagaaatttcttttcataaattggtaattcgtacatcatttataggcgttagctataaccttttagttagtgaatacaatactttttgctattattatgtaacttttagatatgaatttactttcaaaaaaaaaaaggatcgatgttggttatcaactaaggaccaaccactttggacgtctcaccactaagttaaataaatcactttgttctcgaaaaaaaccccaaaagtgttaaaatgcttttcatatcataatcaaacaacgtgattaataaaatctattaagttaatagaagtagggaataaatcgggcaaaagaatttgatacaaaccaaaccggtcaaaaaagctagtattcatataaatggactatacaagttaataccagctagcagaaattaaatagtttattaagttgattacaaaacaattcctcatttaaaaaaagttaattgtaatcaagagatcttttgcttctaattgatcagacgaggacccctcttatttattttctttttcatataagattttgaatagatatagggaaatcttgttcactctttatctacttcaaattgcatgcattttaagaattctctttgtatgcaaacttcagtatttatgattgacataaatcaatattcatatcttcgataaagttaataactctcctaatacttatgaatatctcttcctttacaaccctataaaaccccccactatagctaccttcataattcatcttagagtaccaaccctaaatttcttagtgattaaca

SEQ ID NO: 2 - DNA, 27 bp, synthetic construct
  source 1..27: mol_type = other DNA
  misc_feature 1..27: note = FAD2 TS primer
tttagtcccttatttctcatggaaaat

SEQ ID NO: 3 - DNA, 17 bp, synthetic construct
  source 1..17: mol_type = other DNA
  misc_feature 1..17: note = Forward Primer
tgattgctcacgagtgt

SEQ ID NO: 4 - DNA, 20 bp, synthetic construct
  source 1..20: mol_type = other DNA
  misc_feature 1..20: note = Reverese Primer
cagagacattgaaggctaaa

SEQ ID NO: 5 - DNA, 22 bp, synthetic construct
  source 1..22: mol_type = other DNA
  misc_feature 1..22: note = Reference probe
ccgtgatgaagtgtttgtccca

SEQ ID NO: 6 - DNA, 21 bp, synthetic construct
  source 1..21: mol_type = other DNA
  misc_feature 1..21: note = Drop-off probe
ctcatggaaaataagccatcg

SEQ ID NO: 7 - DNA, 20 bp, synthetic construct
  source 1..20: mol_type = other DNA
  misc_feature 1..20: note = HT-19-001
aagaagaagaggaaggtggg

SEQ ID NO: 8 - DNA, 21 bp, synthetic construct
  source 1..21: mol_type = other DNA
  misc_feature 1..21: note = HT-22-007
gagaggtcagcatcagcatac

SEQ ID NO: 9 - DNA, 19 bp, synthetic construct
  source 1..19: mol_type = other DNA
  misc_feature 1..19: note = HT-21-080
cctcattgcatggccaatc

SEQ ID NO: 10 - DNA, 24 bp, synthetic construct
  source 1..24: mol_type = other DNA
  misc_feature 1..24: note = HT-21-081
ccagagacattgaaggctaaatac